(19) United States
(12) Patent Application Publication
(10) Pub. No.: US 2015/0147403 A1
(43) Pub. Date: May 28, 2015

(54) DERMATOLOGICAL COMPOSITION COMPRISING OLEOSOMES AND RETINOIDS, PROCESS FOR PREPARING THE SAME AND USE THEREOF

(71) Applicant: GALDERMA RESEARCH & DEVELOPMENT, Biot (FR)

(72) Inventor: Amel Djedour, Antibes (FR)

(21) Appl. No.: 14/404,936

(22) PCT Filed: May 30, 2013
(86) PCT No.: PCT/EP2013/061196

§ 371 Date: Dec. 1, 2014

Related U.S. Application Data
(60) Provisional application No. 61/654,687, filed on Jun. 1, 2012.

(30) Foreign Application Priority Data
Jun. 1, 2012 (FR) 1255090

(51) Int. Cl. A61K 31/4015; A61K 9/00; A61K 9/107

(52) U.S. Cl. CPC A61K 31/4015; A61K 9/107; A61K 9/0014
USPC 424/498; 514/424

(57) ABSTRACT

Compositions including at least 3″-tert-butyl-4′-(2-hydroxy-ethoxy)-4″-pyrrolidin-1-yl-[1,1′;3,1″]-terphenyl-4-carboxylic acid are described. The compositions can be oil-in-water emulsions made up of oily liquid globules each provided with a lamellar liquid crystal coating dispersed in an aqueous phase; a retinoid is either in the oily liquid globules or in the aqueous phase; each oily liquid globule is individually coated with a unilamellar or oligolamellar layer, obtained from at least one lipophilic surfactant, at least one hydrophilic surfactant and at least one separate anionic surfactant, and the coated oily liquid globules have a mean diameter lower than 800 nm.

The present invention relates to a pharmaceutical composition, especially a dermatological composition, based on a retinoid, said retinoid being present in dissolved form in oleosomes dispersed in an aqueous phase, or directly dissolved in the aqueous phase. The invention also relates to the process for preparing this composition and to the use thereof for treating dermatological complaints, in particular acne, ichthyosis, ichthyosiform conditions, palmoplantar hyperkeratosis or psoriasis, preferentially acne.

Retinoids are active agents commonly used in dermatology, but the majority are known as being irritant active principles. It is therefore essential to find compositions for maintaining the biological activity and thus the efficacy of a retinoid, while at the same time minimizing its irritant nature.

The search for novel formulations for maintaining the pharmaceutical activity of a retinoid, while at the same time improving its tolerance, is therefore of ongoing interest.

To this end, it has been proposed to improve the topical tolerance of retinoids by adding anti-irritant compounds to the composition.

Patent application FR 2 894 820 discloses, for example, galenical formulations using anti-irritants such as allantoin or disodium edetate in combination with a particular retinoid, adapalene.

Other techniques propose dispersing the retinoid, in free form or by adsorbing it onto particles, so as to improve its tolerance. For example, patent U.S. Pat. No. 5,955,109 proposes to incorporate a retinoid into porous microspheres (Microsponge®) in order to reduce the release of the retinoid into the layers of the skin, which gives rise to a decrease in the level of irritation by controlling the release kinetics of the active agent through the skin. In point of fact, such a system comprising the retinoid in dispersed form involves the formulation of a larger amount of active agent in order to maintain its biological activity. It is therefore necessary to have available formulations that optimize the amount of retinoid with respect to its efficacy.

To do this, formulating a retinoid in dissolved form is the most advantageous solution in terms of costs. Specifically, dissolving a retinoid in a formulation makes it possible to use a smaller amount thereof than in dispersed form. However, the tolerance of the formulation is then reduced, since the active agent can be released more quickly, which facilitates its penetration into the skin.

The prior art, and especially patent U.S. Pat. No. 5,650,171, describes the formulation of retinoids, in particular tretinoin, with polyol prepolymers in large contents (>10%), which complicates the formulation.

Another solution consists in including the retinoid in liposomes. Liposomes comprise phospholipids in which the active agent becomes seated. However, these phospholipids are difficult to formulate, especially at an industrial level, and to stabilize, especially at high temperatures, for instance at 40° C.

Finally, the prior art proposes to dissolve the retinoid, which is generally hydrophobic, in alcoholic solvents, in order generally to obtain an aqueous-alcoholic gel. The amounts of solvent used are then incompatible with the pathologies for which retinoids are effective, namely dermatological pathologies. Notably, the formulation Aberel® from Janssen-Cilag for treating acne (based on 0.025% tretinoin in gel form and comprising a large amount of alcohol) was withdrawn from the market because it was poorly tolerated.

There is thus a need for a stable formulation comprising the retinoid in dissolved form, which is sparingly irritant (and thus well tolerated), which allows controlled release and good penetration of the active agent, which is acceptable as regards its organoleptic properties, and which is readily manufacturable and industrializable.

The problem that the present invention proposes to solve here is thus that of designing a physically and chemically stable composition comprising at least one retinoid, for the treatment of dermatological pathologies, more particularly acne, said retinoid being in dissolved form, the composition according to the invention being readily industrializable and needing to improve the tolerance of the active principle while at the same time being easy to use and being cosmetically acceptable for application to any area of the body that might be affected by the pathology.

According to the present invention, the retinoid must be in a dissolved form in a stable composition. Many retinoids often present solubilization difficulties. The retinoids according to the invention, and especially the retinoid preferentially used, have low solubility, thus limiting their incorporation into the standard vehicles, and making it difficult to obtain a stable composition. Moreover, the addition of a solubilizer to topical formulations often increases the irritant power of the formulations.

The present invention thus relates to the formulation of oleosomes which can improve the cutaneous tolerance of retinoids in the treatment of dermatological pathologies, especially acne, ichthyosis, ichthyosiform conditions, palmoplantar hyperkeratosis or psoriasis, preferentially acne.

The Applicant has thus discovered, surprisingly, that compositions comprising at least one retinoid in dissolved form, either in oleosomes dispersed in an aqueous phase, or directly dissolved in the aqueous phase, not requiring any polymer and/or requiring little or no organic solvent, ensure the stability (chemical and physical) of the active agent and satisfactory tolerance of the composition. The compositions according to the invention may also promote the cutaneous penetration of the active agent, which is useful in the treatment of dermatological complaints, especially acne. In another preferred embodiment according to the invention, the Applicant has discovered that the tolerance of the active agent is improved when it is dissolved in the oily inner phase of the oleosomes, while at the same time obtaining controlled release and good penetration of the active agent into the skin.

The term “oleosomes” refers to oily liquid globules individually coated with a monolamellar or oligolamellar liquid crystal layer obtained from at least one lipophilic surfactant, at least one hydrophilic surfactant and at least one distinct anionic surfactant. These oleosomes are dispersed in the aqueous phase (continuous phase).

According to the invention, the term “physical stability” refers to a composition whose physical properties such as the organoleptic properties, pH and viscosity are stable over time and under various temperature conditions (for example 4° C., room temperature and 40° C.).

The term “room temperature” means a temperature between 15 and 25° C.

According to the invention, the term “chemical stability” refers to a composition in which the active principle is chemically stable over time, irrespective of the temperature condition: 4° C., room temperature, 40° C.

One subject of the present invention is thus especially a composition, in particular a pharmaceutical composition, comprising at least one particular retinoid, namely 3″-tert-butyl-4′-(2-hydroxyethoxy)-4″-pyrrolidin-1-yl-[1,1′;3′,1″]-terphenyl-4-carboxylic acid, as pharmaceutical active agent, characterized in that:
said composition is an oil-in-water emulsion formed from oily liquid globules each provided with a lamellar liquid-crystal coating and dispersed in an aqueous phase,
said retinoid is dissolved either in the oily liquid globules or in the aqueous phase of the oil-in-water emulsion,
each oily liquid globule is individually coated with a monolamellar or oligolamellar layer obtained from at least one lipophilic surfactant, at least one hydrophilic surfactant and at least one distinct anionic surfactant.

In a preferred embodiment, a subject of the present invention is a composition, especially a pharmaceutical composition, comprising:
an oil-in-water emulsion formed from oily liquid globules each provided with a lamellar liquid-crystal coating and dispersed in an aqueous phase,
each oily liquid globule is individually coated with a monolamellar or oligolamellar layer obtained from at least one lipophilic surfactant, at least one hydrophilic surfactant and at least one distinct anionic surfactant, characterized in that said composition comprises 3″-tert-butyl-4′-(2-hydroxyethoxy)-4″-pyrrolidin-1-yl-[1,1′;3′,1″]-terphenyl-4-carboxylic acid dissolved in the oily liquid globules.

In another embodiment according to the invention, the 3″-tert-butyl-4′-(2-hydroxyethoxy)-4″-pyrrolidin-1-yl-[1,1′;3′,1″]-terphenyl-4-carboxylic acid is partially dissolved in the oily liquid globules and partially dissolved in the aqueous outer phase of the composition.

A subject of the present invention is also the use of a composition comprising at least one retinoid:
said composition being an oil-in-water emulsion comprising oily liquid globules each provided with a lamellar liquid-crystal coating and dispersed in an aqueous phase,
the retinoid being dissolved either in the oily liquid globules or in the aqueous phase of the oil-in-water emulsion,
each oily liquid globule being individually coated with a monolamellar or oligolamellar layer obtained from at least one lipophilic surfactant, at least one hydrophilic surfactant and at least one distinct anionic surfactant, and the coated oily liquid globules having a mean diameter of less than 800 nm,
   for improving the tolerance of said retinoid.

Preferably, the retinoid employed in this use is 3″-tert-butyl-4′-(2-hydroxyethoxy)-4″-pyrrolidin-1-yl-[1,1′;3′,1″]-terphenyl-4-carboxylic acid.

Another subject of the present invention is the use of the composition defined previously for improving the tolerance of the retinoid when it is dissolved in the oily inner phase of the composition, while at the same time controlling the release and penetration of the active agent.

Preferably, the retinoid used is 3″-tert-butyl-4′-(2-hydroxyethoxy)-4″-pyrrolidin-1-yl-[1,1′;3′,1″]-terphenyl-4-carboxylic acid.

According to another embodiment, the invention relates to the use of a composition as defined previously for improving the cutaneous penetration of the retinoid, said composition not containing any pro-penetrating agent.

Preferably, the retinoid used is 3″-tert-butyl-4′-(2-hydroxyethoxy)-4″-pyrrolidin-1-yl-[1,1′;3′,1″]-terphenyl-4-carboxylic acid.

The invention also relates to the use of a composition as defined previously for obtaining controlled release of the retinoid, characterized in that it is dissolved in the oily liquid globules.

Preferably, the retinoid used is 3″-tert-butyl-4′-(2-hydroxyethoxy)-4″-pyrrolidin-1-yl-[1,1′;3′,1″]-terphenyl-4-carboxylic acid.

The retinoids that may be used in the context of the invention especially comprise all-trans-retinoic acid or tretinoin, 13-cis-retinoic acid or isotretinoin, acitretin, arotinoic acid, retinol, adapalene, tazarotene, retinaldehyde, etretinate and the compounds protected in patent application WO 2006/066 978 such as 3″-tert-butyl-4′-(2-hydroxyethoxy)-4″-pyrrolidin-1-yl-[1,1;3′,1″]-terphenyl-4-carboxylic acid, the compounds of patent application FR 05/12367 including 2-hydroxy-4-[3-hydroxy-3-(5,6,7,8-tetrahydro-5,5,8,8-tetramethyl-2-naphthyl)-1-propynyl]benzoic acid or an enantiomer thereof, the compounds of patent application WO 05/56516 including 4′-(4-isopropylaminobutoxy)-3′-(5,5,8,8-tetramethyl-5,6,7,8-tetrahydronaphthalen-2-yl)-biphenyl-4-carboxylic acid, the compounds of patent application PCT/EP04/014809 including 4-{3-hydroxy-3-[4-(2-ethoxyethoxy)-5,5,8,8-tetramethyl-5,6,7,8-tetrahydronaphthalen-2-yl]prop-1-ynyl}benzoic acid, and the compounds of patent application FR 2 861 069 including 4-[2-(3-tert-butyl-4-diethylaminophenyl)-2-hydroxyiminoethoxy]-2-hydroxybenzoic acid.

3″-tert-Butyl-4′-(2-hydroxyethoxy)-4″-pyrrolidin-1-yl-[1,1′;3′,1″]-terphenyl-4-carboxylic acid as protected in patent application WO 2006/066 978 is particularly preferred, and will be referred to as “compound A” in the rest of the present patent application.

The composition according to the invention comprises between 0.00001% and 1% and preferably from 0.0001% to 0.5% by weight of at least one retinoid relative to the total weight of the composition, and preferentially the composition according to the invention contains from 0.001% to 0.05% by weight of a retinoid relative to the total weight of the composition. In a preferred embodiment according to the invention, the composition comprises between 0.001% and 0.05% and more particularly between 0.003% and 0.03% by weight of 3″-tert-butyl-4′-(2-hydroxyethoxy)-4″-pyrrolidin-1-yl-[1,1;3′,1″]-terphenyl-4-carboxylic acid relative to the total weight of the composition.

According to the present invention, the retinoid is dissolved either in the oily liquid globules or in the aqueous phase of the oil-in-water emulsion, Preferably, the retinoid is dissolved in the oily liquid globules (these oily liquid globules are also referred to as the “inner phase” or the “oily inner phase” in the present invention). Specifically, the Applicant has discovered, surprisingly, that internalization of the active agent in the oily liquid globules of the composition ensures better tolerance of the composition, while at the same time giving the active agent controlled release and good penetration, comparable to the reference formulations in which the active agent is in the outer aqueous phase.

Furthermore, when the retinoid is dissolved in the inner phase of the invention, it is possible to limit or to dispense with the use of a pro-penetrating agent. Specifically, although often essential for improving the penetration of pharmaceutical active agents and thus their efficacy, pro-penetrating agents may have the drawback of being irritant. Thus, according to a preferred embodiment, when the retinoid is dissolved in the oily liquid globules, the composition contains substantially no pro-penetrating agent, and is preferably free of pro-penetrating agent.

The term “composition comprising substantially no pro-penetrating agent” means a composition containing less than 0.5%, preferably less than 0.3% and preferably less than 0.1% by weight of pro-penetrating agent.

The term “pro-penetrating agent” means alcohols such as ethanol; glycols, for instance propylene glycol; glycol ethers, for instance PPG-15 stearyl ether; N-methyl-2-pyrrolidone; dimethyl sulfoxide, or dimethyl isosorbide.

The composition according to the invention may be incorporated into a pharmaceutically acceptable vehicle, such as a gel, a solution or an emulsion, for instance a cream or a lotion, or a sprayable or non-sprayable, pressurized or non-pressurized foam.

According to the present invention, the composition comprises oleosomes rather than lipid nanospheres. Specifically, oleosomes are oily liquid globules coated with a non-polymeric envelope (i.e. formed from at least one lipophilic surfactant, at least one hydrophilic surfactant and at least one distinct anionic surfactant), as opposed to lipid nanospheres, which are matrix particles, i.e. all of the mass of which is solid at room temperature.

The oleosomes (oily liquid globules) according to the present invention have a mean size of less than 800 nm and preferably less than 500 nm. The mean diameter of the oleosomes and the particle size distribution may be determined by DLS (dynamic light scattering) using a particle size analyzer such as a Nano ZS Zetasizer (Malvern Instruments), as explained in the examples.

The particle size distribution of the oleosomes may also be controlled in the course of manufacture, during the preparation of the “oleosome” phase, so as to ensure the fineness of the emulsion, by sampling.

Oleosomes thus consist of oil globules each provided with a lamellar liquid-crystal coating, the coating being a monolamellar or oligolamellar layer obtained from at least one lipophilic surfactant, at least one hydrophilic surfactant and at least one distinct anionic surfactant; they are dispersed in the aqueous phase.

The term “oligolamellar layer” means a layer comprising from 2 to 5 lipid leaflets, as described in patent EP 0 641 557.

Preferably, the lipophilic surfactant used for forming the oleosomes has an HLB of between 2 and 5.

As lipophilic surfactant with an HLB of between 2 and 5 that may be used according to the invention, mention will be made of sorbitan esters, such as sorbitan monostearate (HLB=4.7) sold under the name Span® 60 by the company Croda, glyceryl esters such as the glyceryl monostearate sold under the name Cutina® GMSVPH (HLB=3.8) by the company BASF, low-HLB sugar esters such as the sucrose dilaurate sold under the name Surfhope® C-1205 (HLB=5) or the sucrose tristearate sold under the name Surfhope® C-1803 (HLB=3) by the company Gattefossé, or alternatively polyoxyethylene stearyl ether comprising 2 oxyethylene units (2 OE). Preferably, the lipophilic surfactant with an HLB of between 2 and 5 is chosen from sucrose distearate, diglyceryl distearate, tetraglyceryl tristearate, decaglyceryl decastearate, diglyceryl monostearate, sorbitan monostearate, sorbitan tristearate, 15 diethylene glycol monostearate, the glyceryl ester of palmitic and stearic acids, polyoxyethylene monostearate comprising 2 oxyethylene units (2 OE), polyoxyethylene stearyl ether comprising 2 oxyethylene units (2 OE), i.e. Steareth-2, glyceryl monobehenate and dibehenate and pentaerythrityl tetrastearate.

Preferably, the hydrophilic surfactant used for forming the oleosomes has an HLB of between 8 and 12.

Nonlimiting examples of hydrophilic surfactants with an HLB of between 8 and 12 that may be mentioned include polyoxyethylene glycol esters such as glyceryl stearate and PEG-100 stearate sold under the name Arlacel® 165 FL (HLB=11) by the company Croda, PEG-6 stearate and PEG-32 stearate sold under the name Tefose® 1500 (HLB=10) by the company Gattefossé, polyoxyethylene sorbitan esters and high-HLB sugar esters such as the sucrose stearate sold under the name Surfhope® C-1811 (HLB=11) by the company Gattefossé, or alternatively polyoxyethylene stearyl ether comprising 10 oxyethylene units.

Preferably, the hydrophilic surfactant with an HLB of between 8 and 12 is chosen from polyoxyethylene sorbitan monostearate 4 OE, polyoxyethylene sorbitan tristearate 20 OE, polyoxyethylene monostearate 8 OE, hexaglyceryl monostearate, polyoxyethylene monostearate 10 OE, polyoxyethylene stearyl ether 10 OE, i.e. Steareth-10, polyoxyethylene distearate 12 OE and polyoxyethylene methylglucose distearate 20 OE.

The term “distinct anionic surfactant” means an anionic surfactant other than the lipophilic surfactant and the hydrophilic surfactant forming the coating of the oleosomes.

Preferably, the distinct anionic surfactant is a fatty acid or a fatty acid ester, said fatty acid comprising at least one saturated fatty chain containing more than 12 and preferably between 16 and 22 carbon atoms.

The distinct anionic surfactant may also be an ionic amphiphilic lipid. The ionic amphiphilic lipid is preferably chosen from the group comprising neutralized anionic lipids, amphoteric lipids and alkylsulfonic derivatives.

The neutralized anionic lipids are chosen in particular from:
alkali metal salts of dicetyl phosphate, and in particular the sodium and potassium salts;
alkali metal salts of dimyristyl phosphate, and in particular the sodium and potassium salts;
alkali metal salts of cholesteryl sulfate, and in particular the sodium salt;
alkali metal salts of cholesteryl phosphate, and in particular the sodium salt;
monosodium and disodium salts of acylglutamic acids, and in particular the monosodium and disodium salts of N-stearoylglutamic acid, or
the sodium salt of phosphatidic acid, for instance the sodium stearoyl glutamate sold under the name Eumulgin SG by Cognis.

The amphoteric lipids are chosen in particular from phospholipids and especially pure soybean phosphatidylethanolamine.

The alkylsulfonic derivatives are advantageously the compounds of formula:

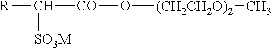

in which R represents the radicals C₁₆H₃₃ and C₁₈H₃₇ taken alone or as a mixture and M is an alkali metal, preferably sodium.

More preferentially, the distinct anionic surfactant is chosen from stearic acid, palmitic acid, arachidic acid and behenic acid.

In the case of using a fatty acid, and in order to enable the lamellar system to form, the pH of the composition according to the invention is high and should be greater than 7 (basic). The reason for this is that at a pH above 7, the fatty acids are anionic.

The basic agent for neutralizing the fatty acid present in the oleosomes may be triethanolamine, sodium hydroxide, lysine or arginine.

Preferably, the surfactants are mixed in the following proportions to form the oleosomes:
lipophilic surfactant (HLB between 2 and 5): 45-50% by weight of the mixture;
hydrophilic surfactant (HLB between 8 and 12): 30-35% by weight of the mixture; and
distinct anionic surfactant: 20-25% by weight of the mixture.

Preferably, the total amount of surfactants present in a composition obtained via the process according to the invention is between 3% and 4% of the total weight of the composition.

An oleosome according to the invention consists of a coating and of an oily liquid globule in which the retinoid may be dissolved. Preferably, the retinoid, and more particularly 3″-tert-butyl-4′-(2-hydroxyethoxy)-4″-pyrrolidin-1-yl-[1,1;3′,1″]-terphenyl-4-carboxylic acid, is in dissolved form in the oily liquid globules.

The composition of the oily liquid globules is essential for the stability of the retinoid. These oily liquid globules must, of course, be compatible with the retinoid to be dissolved, and must be capable of dissolving the retinoid.

Preferably, the oily liquid globules comprise at least one oily solvent chosen from phenoxyethanol, glycol ethers, polyethoxylated fatty acids, triglycerides and oils containing the same, and fatty acid esters.

The term “oily solvent” means any water-immiscible material of natural, animal or synthetic origin. The oily solvent is a solvent in which the active agent is dissolved and chemically stable. It may be present in a concentration ranging from 0.01% to 20% by weight.

Among the triglycerides and oils containing the same, mention may be made in a nonlimiting manner of octanoic acid triglycerides or caprylic/capric acid triglycerides, such as those sold by the company Stéarineries Dubois or those sold under the names Miglyol® 810, 812 and 818 by the company Sasol.

Among the glycol ethers, mention may be made in a nonlimiting manner of polypropylene glycol ethers such as the PPG-15 stearyl ether sold under the name Arlamol® PS15E by the company Croda.

Among the fatty acid esters, mention may be made in a nonlimiting manner of the diisopropyl adipate sold under the name Crodamol® DA by the company Croda, or cetearyl isononanoate sold under the name Kollicream® CI by the company BASF.

In the case where the active agent of the composition according to the invention is Compound A, the oily solvent may be, for example, the phenoxyethanol sold under the name Phenoxetol® by Clariant, present in a concentration ranging from 0.2% to 5% and preferentially from 0.5% to 2% by weight.

In addition to this or these oily solvent(s), the oily liquid globules may also comprise one or more fatty substances that are liquid or semiliquid at room temperature and that cannot dissolve the retinoid. These compounds are especially mineral oils, natural or synthetic plant oils, animal oils or silicone oils.

The fatty phase of the invention may also comprise:
one or more mineral oils, for instance liquid paraffins of different viscosities, for instance Marcol® 152, Marcol® 52 or Primol® 352 sold by Univar,
one or more plant oils, among which mention may be made of sweet almond oil, palm oil, soybean oil, sesame oil, sunflower oil, hydrogenated castor oil or coconut oil,
one or more synthetic oils, among which mention may be made of apricot kernel oil PEG-6 ester (Labrafil® M1944CS), propylene glycol laurate (Lauroglycol® FCC), propylene glycol monocaprylate (Capryol® 90) sold by Gattefossé, esters such as cetearyl isononanoate, for instance the product sold under the name Kollicream® CI by the company BASF France, and isopropyl palmitate, for instance the product sold under the name Crodamol® IPP by the company Croda,
one or more animal oils, among which mention may be made of lanolin, squalene, fish oil, mink oil, with, as a derivative, the squalane sold under the name Cosbiol® by the company Laserson,
one or more silicone oils for improving the properties of the formula on application, such as cyclomethicone (St-Cyclomethicone® 5NF) or dimethicone (Q7 9120 Silicon Fluid® with a viscosity from 20 cSt to 12 500 cSt from Dow Corning),
one or more fatty-phase thickeners of fatty alcohol type, such as cetyl alcohol (Crodacol® C70 sold by Croda/Lanette 16 sold by BASF, but also Kolliwax® CA sold by BASF), cetearyl alcohol (Crodacol® 1618 sold by Croda, Tego Alkanol® 1618 sold by Evonik, but also Kolliwax® CSA Pharma sold by BASF), stearyl alcohol (Crodacol® S95 sold by Croda, Kolliwax® SA sold by BASF, but also Tego Alkanol® 18 sold by Evonik), but also behenyl alcohol (Lanette® 22 sold by BASF, Nacol® 22-98 sold by Sasol, but also Behenyl Alcohol 65 80 sold by Nikko Chems), or of carnauba wax type sold by Baerlocher, but also the beeswax sold under the name Cerabeil Blanchie DAB® sold by Univar, and glyceryl tribehenate such as Compritol® 888 sold by Gattefossé. In this case, a person skilled in the art will adjust the heating temperature of the preparation according to the presence or absence of these solids.

Other oils or fatty substances may be added to the fatty phase of the composition in a varied manner by a person skilled in the art in order to prepare a composition having the desired properties, for example in terms of consistency or texture.

Preferably, the composition according to the invention has a ratio between the total amount of surfactants and the total amount of oily phase constituting the oily liquid globules of between 10% and 25%. With such a ratio, the oleosomes have an acceptable size, with a lamellar liquid-crystal coating around the droplets of monolamellar or oligolamellar oil.

The oleosomes are dispersed in an aqueous phase, which is the continuous phase. The latter phase comprises water. This water may be demineralized water, a floral water such as cornflower water, or a natural spring water or mineral water, chosen, for example, from Vittel water, Vichy basin water, Uriage water, Roche Posay water, Bourboule water, Enghien-les-Bains water, Saint Gervais-les-Bains water, Néris-les-Bains water, Allevard-les-Bains water, Digne water, Maizières water, Neyrac-les-Bains water, Lons-le-Saunier water, Eaux Bonnes water, Rochefort water, Saint Christau water, Fumades water, Tercis-les-Bains water, Avène water or Aix-les-Bains water.

The water may be present in a content of between 25% and 90% by weight and preferably between 50% and 90% by weight relative to the total weight of the composition.

The aqueous phase may contain, in a nonlimiting manner, one or more polyols, and various types of additives.

According to a preferred embodiment, the composition according to the invention has a ratio between the aqueous phase and the fatty phase (coated oily liquid globules) as follows: % fatty phase/(% fatty phase+% aqueous phase) between 0.40 and 0.55, preferably between 0.45 and 0.50, and preferably equal to 0.48.

The composition according to the invention may be incorporated into a pharmaceutically acceptable vehicle, such as a gel, a solution or an emulsion, for instance a cream or a lotion, or a foam.

When the pharmaceutically acceptable vehicle is a gel, the composition according to the invention is dispersed in a hydrophilic phase which comprises at least one gelling agent. This gelling agent may be a cellulose derivative chosen from semi-synthetic cellulose-based gelling agents, such as methylcellulose, ethylcellulose or hydroxypropylmethylcellulose sold by the company Colorcon under the name Methocel® (for example: Methocel® E4M), hydroxyethylcellulose sold by the company Ashland under the name Natrosol® (for example: Natrosol® 250 HHX), carboxymethylcellulose, hydroxymethylcellulose and hydroxypropylcellulose, taken alone or as a mixture. The gelling agent may also be chosen from natural gums such as gum tragacanth, guar gum, acacia gum, gum arabic, xanthan gum, starch and derivatives thereof, copolymers of polyacrylic acid, for instance the carbomer products sold by the company Lubrizol (i.e. Carbomer 980), and of methyl methacrylate, carboxyvinyl polymers, polyvinylpyrrolidones and derivatives thereof, polyvinyl alcohols, biopolymers such as sodium alginate, pectin, dextrin, chitosan or sodium hyaluronate and derivatives thereof, taken alone or as a mixture. The gelling agent may also be chosen from the compound Sepigel® 305 consisting of a polyacrylamide/C13-C14 isoparaffin/laureth-7 mixture, or Simulgel® 600PHA or Sepineo® P600, namely sodium acryloyldimethyltaurate copolymer/isohexadecane/polysorbate 80, these two products being sold by the company SEPPIC. Preferably, Simulgel® 600PHA is used.

The gelling agent is especially used in a concentration of between 0.1% and 10% by weight and preferably between 0.1% and 4% by weight relative to the total weight of the composition.

When the pharmaceutically acceptable vehicle is a solution, the composition according to the invention is dispersed in a vehicle composed of an aqueous phase (as defined previously in the present invention).

When the pharmaceutically acceptable vehicle is a cream or a lotion, the composition according to the invention is dispersed in a vehicle composed of an aqueous phase and of a fatty phase optionally comprising at least one surfactant or emulsifier.

The composition according to the invention may also contain additives or combinations of additives, such as:
co-surfactants such as fatty alcohols;
stabilizers;
humectants;
humidity regulators;
pH regulators;
osmotic pressure modifiers;
chelating agents;
preserving agents;
UV-A and UV-B screening agents;
and antioxidants.

Needless to say, a person skilled in the art will take care to select the optional compound(s) to be added to these compositions such that the advantageous properties intrinsically associated with the present invention are not, or are not substantially, adversely affected by the envisaged addition.

These additives may be present in the composition from 0 to 40% by weight relative to the total weight of the composition.

The pharmaceutical composition that may be used according to the invention is intended for treating the skin and may be administered topically, parenterally or orally. Preferably, the composition is administered topically. The term “topical route” means application to the skin or mucous membranes, and also to the hair or the scalp.

Via the oral route, the pharmaceutical composition may be in liquid or pasty form, and more particularly in the form of gel capsules, coated tablets or syrups.

Via the parenteral route, the composition may be in the form of suspensions for perfusion or for injection.

Via the topical route, the composition may be in liquid or pasty form, and more particularly in the form of creams, milks, pomades, impregnated pads, syndets, wipes, gels, sprays, foams, lotions, sticks, shampoos or washing bases.

The composition in oil-in-water emulsion form comprising the oily liquid globules according to the present invention may be obtained by simple mixing of two phases, namely:
an aqueous phase, heated to a temperature not above 75° C., preferably between 65° C. and 75° C.,
an oily phase, heated to a temperature not above 75° C., preferably between 65° C. and 75° C., and containing the mixture of surfactants (i.e. lipophilic surfactant, hydrophilic surfactant and distinct anionic surfactant).

Emulsification is obtained either using a high-pressure homogenizer (HPH) or by simple mixing of the two heated phases using a turbomixer. In the latter case, the ratio between the aqueous phase and the fatty phase is preferably as follows:

% fatty phase/(% fatty phase+% aqueous phase)=0.48.

The composition according to the invention may be used as a medicament.

In particular, a subject of the invention is also a composition for use in the treatment of dermatological complaints, especially human complaints, as defined below:

1) dermatological conditions associated with a keratinization disorder relating to cell differentiation and proliferation, in particular for treating common acne, comedonal acne, polymorphic acne, acne rosacea, nodulocystic acne, acne conglobata, senile acne, secondary acne such as solar acne, acne medicamentosa or occupational acne;
2) keratinization disorders, in particular ichthyosis, ichthyosiform conditions, lamellar ichthyosis, Darier's disease, palmoplantar keratoderma, leukoplakia, pityriasis rubra pilaris and leukoplakiform conditions, cutaneous or mucosal (buccal) lichen;
3) dermatological complaints with an inflammatory immunoallergic component, with or without a cell proliferation disorder, and in particular all forms of psoriasis, whether cutaneous, mucosal or ungual, and even psoriatic arthritis, or else atopic dermatitis and the various forms of eczema;
4) skin disorders caused by exposure to UV radiation, and also for repairing or combating skin aging, whether it is photo-induced or chronological, or for reducing actinic keratoses and pigmentations, or any pathological condition associated with chronological or actinic aging, such as xerosis, pigmentations and wrinkles;
5) any condition associated with benign dermal or epidermal proliferations, whether or not they are of viral origin, such as common warts, flat warts, molluscum contagiosum and epidermodysplasia verruciformis, or oral or florid papillomatoses;
6) dermatological disorders such as immune dermatoses, for instance lupus erythematosus, bullous immune diseases and collagen diseases, such as scleroderma;
7) stigmata of epidermal and/or dermal atrophy induced by local or systemic corticosteroids, or any other form of cutaneous atrophy;
8) cicatrization disorders, or for preventing or repairing stretch marks, or else for promoting cicatrization;
9) in the treatment of any condition of fungal origin at the cutaneous level, such as tinea pedis and tinea versicolor;
10) pigmentation disorders, such as hyperpigmentation, melasma, hypopigmentation or vitiligo;
11) cutaneous or mucosal cancerous or precancerous conditions, such as actinic keratoses, Bowen's disease, in-situ carcinomas, keratoacanthomas and skin cancers such as basal cell carcinoma (BCC), squamous cell carcinoma (SCC) and cutaneous lymphomas such as T lymphoma.

More preferentially, the composition according to the invention is a composition for use in the treatment of acne, ichthyosis, ichthyosiform conditions, palmoplantar hyperkeratosis or psoriasis.

The composition according to the invention is in effect suitable for use in the treatment of dermatological complaints, especially human complaints, and preferably for treating acne.

The present invention also relates to the use of a composition comprising at least one retinoid, for improving the tolerance of the retinoid, said composition being an oil-in-water emulsion comprising oily liquid globules each containing a lamellar liquid-crystal coating and dispersed in an aqueous phase, the retinoid being dissolved either in the oily liquid globules or in the aqueous phase of the oil-in-water emulsion, each oily liquid globule being individually coated with a monolamellar or oligolamellar layer obtained from at least one lipophilic surfactant, at least one hydrophilic surfactant and at least one distinct anionic surfactant, and the coated oily liquid globules having a mean diameter of less than 800 nm.

The use of the composition according to the invention is also intended to improve the tolerance of the retinoid when it is dissolved in the oily inner phase of the composition, while at the same time controlling the release and penetration of the active agent.

In another embodiment, the invention thus relates to the use of a composition according to the invention for improving the cutaneous penetration of the retinoid, said composition not containing any pro-penetrating agent.

The invention also relates to the use of a composition according to the invention for obtaining controlled release of the retinoid, characterized in that it is dissolved in the oily liquid globules.

Various composition formulations according to the invention will now be given, as illustrations and with no limiting nature.

In the various examples listed hereinbelow:
phase A denotes the oily phase constituting the oily liquid globule,
phase B denotes the aqueous phase,
phase C denotes the phase consisting of the gelling agent(s) and other additives, not incorporated into the oily phase (A) or aqueous phase (B).

### EXAMPLE 1

### Solubility Data for Compound A in Various Oily and Aqueous Solvents

The object of this preformulation study is to identify lipophilic and hydrophilic solvents in which compound A is soluble, and in which it is chemically stable.

The stability of the active agent was evaluated by liquid chromatography coupled to a UV detector (HPLC-UV).

| | Maximum | |
|---|---|---|
| INCI name (trade name) | solubility (% m/m) | Stability* |
| Phenoxyethanol | 1.957 | 6 months RT / 40° C. |
| Absolute ethanol | 0.92 | 3 months RT / 40° C. |
| Propylene glycol | 0.11 | 3 months RT / 40° C. |
| Propylene glycol / absolute | 1.08 | 6 months RT / 40° C. |
| ethanol (20/80) | | |
| PPG-15 stearyl ether | 0.292 | 6 months RT / 40° C. |
| (Arlamol E PSE15) | | |
| Caprylic/capric acid | 0.019 | 6 months RT / 40° C. |
| triglycerides (Miglyol 812N) | | |

*:RT:Room temperature

This study moreover makes it possible to confirm that the active agent is ideally dissolved in compounds of alcohol or glycol type and is sparingly soluble in lipophilic solvents such as Miglyol 812.

Depending on the phase into which compound A is introduced, the following solvents will thus be used:
ethanol and propylene glycol as a mixture, in the compositions comprising compound A in the aqueous phase,
PPG-15 stearyl ether, Miglyol and phenoxyethanol as a mixture, in the compositions comprising compound A in the oily liquid globules.

### EXAMPLE 2

### Compositions According to the Invention Comprising Compound A in the Oily Liquid Globules

i) Compositions 1 and 1′:

Composition 1 comprises the following ingredients:

| Phase | Ingredients | Content (% m/m) |
|---|---|---|
| A | Steareth-10 | 1.10 |
| A | Stearic acid | 0.70 |
| A | Stearyl alcohol | 0.25 |
| A | Steareth-2 | 1.60 |
| A | Capric/caprylic acid triglycerides | 15.00 |
| A | Phenoxyethanol | 1.00 |
| A | DL-α-Tocopherol | 0.20 |
| A | Compound A | 0.01 |
| B | Disodium edetate | 0.10 |
| B | Glycerol | 5.00 |
| B | Purified water | 43.75 |
| B | Triethanolamine | 0.20 |
| C | Carbomer 980 | 0.50 |
| C | Purified water | 30.00 |
| C | Triethanolamine | 0.60 |

Composition 1′ comprises the following ingredients:

| Phase | Ingredients | Content (% m/m) |
|---|---|---|
| A | Steareth-10 | 1.25 |
| A | Stearic acid | 0.85 |
| A | Stearyl alcohol | 0.25 |
| A | Steareth-2 | 1.80 |
| A | Capric/caprylic acid triglycerides | 15.50 |
| A | Cyclopentasiloxane | 4.00 |
| A | Phenoxyethanol | 1.00 |
| A | DL-α-Tocopherol | 0.20 |
| A | Compound A | 0.01 |
| B | Disodium edetate | 0.10 |
| B | Glycerol | 5.00 |
| B | Methyl paraben | 0.20 |
| B | Purified water | 21.80 |
| B | Triethanolamine | 0.20 |
| C | Acrylamide/Sodium acryloyldimethyltaurate | 4.00 |
| | copolymer & Isohexadecane & Polysorbate 80 | |
| C | Purified water | 43.85 |

It may be noted that compositions 1 and 1′ do not comprise any pro-penetrating agent.

ii) Process for Manufacturing Compositions 1 and 1′:

### A/Preparation of the Emulsion

Weigh out all the elements of the fatty phase (A) in a tared formulation beaker of suitable size, and heat them at 70° C., with magnetic stirring, until a homogeneous mixture is obtained.
Weigh out all the elements of the aqueous phase (B) is part of the water in an additional formulation beaker, and heat them at 70° C., with magnetic stirring, until a homogeneous mixture is obtained.
Pour the aqueous phase (B) rapidly into the fatty phase (A) with vigorous stirring (Polytron® type rotor-stator—maximum speed 13 000 rpm). Keep stirring for 40 minutes.

### B/Dilution of the Emulsion

Stop the Polytron® stirring and place the beaker containing the emulsion in a cold-water bath (crushed ice if possible) to promote rapid cooling.
Next, stir the emulsion slowly (Rayneri deflocculating paddle—200 rpm).
Gradually add the remainder of the water for the aqueous phase (B) precooled to +4° C. so as to further accelerate the cooling.
Keep stirring until the temperature of the emulsion is below 25° C.

### C/Preparation of the Gel

The gel (phase C) may be prepared in advance (during the emulsification):

### For Composition 1:

Disperse the carbomer in the water to swell it with moderate stirring (Rayneri deflocculating paddle—700 rpm).
Add the triethanolamine to neutralize the gel.

### For Composition 2:

Add the water for phase C and then incorporate the acrylamide copolymer with moderate stirring (Rayneri deflocculating paddle—700 rpm).

### D/Gelation of the Oleosomes

After cooling the emulsion to room temperature, add water if necessary to compensate for the losses.
Stir (Rayneri deflocculating paddle—200 rpm) until homogeneous.
Gradually add the gel to the emulsion, with slow stirring (Rayneri deflocculating paddle—200 rpm) and keep stirring until a homogeneous mixture is obtained (if necessary, increase the stirring to 800 rpm maximum for 3 minutes to aid the homogenization, then reduce the speed again).

### EXAMPLE 3

### Composition According to the Invention Comprising Compound A in the Aqueous Phase

Composition 2 comprises the following ingredients:

| Phase | Ingredients | Content (% m/m) |
|---|---|---|
| A | Steareth-10 | 1.10 |
| A | Stearic acid | 0.70 |
| A | Stearyl alcohol | 0.25 |
| A | Steareth-2 | 1.60 |
| A | Capric/caprylic acid | 6.00 |
| | triglycerides | |
| A | Cetostearyl isononanoate | 6.00 |
| A | Cyclopentasiloxane | 3.00 |
| A | DL-α-Tocopherol | 0.20 |
| B | Compound A | 0.01 |
| B | Disodium edetate | 0.10 |
| B | Glycerol | 5.00 |
| B | Purified water | 30.00 |
| B | Triethanolamine | 0.20 |
| B | Phenoxyethanol | 1.00 |
| B | Propylene glycol | 20.00 |
| B | Absolute ethanol | 10.00 |
| C | Carbomer 980 | 1.00 |
| C | Triethanolamine | 1.20 |
| C | Purified water | 12.65 |

It is prepared according to the protocol indicated in Example 2 ii).

### EXAMPLE 4

### Stability Study on Composition 1′ of Example 2 and on Composition B of Example 3

Composition 1′ of Example 2 and composition 2 of Example 3 were placed under various stability conditions: 4° C., room temperature and 40° C., for 3 months.

The size of the oleosomes was monitored during the stability study by DLS (dynamic light scattering) using a Nano ZS Zetasizer particle size analyzer (Malvern Instruments), after diluting the samples in distilled water.

The pH was measured directly in the composition.

Compound A was assayed by HPLC. The results are expressed in mg/g.

The results are given in the table below.

| | Composition 1′ | | | Composition 2 | | |
|---|---|---|---|---|---|---|
| | (oily phase) | | | (aqueous phase) | | |
| | Microscopic appearance | | | | | |
| | Smooth thick | | | Smooth thick | | |
| | white cream | | | white cream | | |
| Assay (mg/g) | RT | 4° C. | 40° C. | RT | 4° C. | 40° C. |
| T0 | 0.48 | — | — | 104.0 | — | — |
| T1M | 0.049 | 0.047 | 0.049 | 102.0 | 100.6 | 102.6 |
| T2M | 0.049 | 0.048 | 0.047 | 105.5 | 105.6 | 104.7 |
| T3M | 0.048 | 0.47 | 0.048 | 102.6 | 103.3 | 111.0 |
| pH | | | | | | |
| T0 | 7.53 | — | — | 6.96 | — | — |
| T1M | 7.45 | 7.62 | 7.37 | 6.89 | 6.89 | 6.79 |
| T2M | 7.38 | 7.84 | 7.29 | ND | ND | ND |
| T3M | 7.42 | 7.81 | 7.27 | ND | ND | ND |

ND: Not done

### Conclusion:

The stability results show that compound A is chemically stable for 3 months, under all the temperature conditions, both when it is dissolved in the aqueous phase and when it is dissolved in the oily liquid globules, i.e. the oleosomes.

### EXAMPLE 5

### Compositions According to the Invention Comprising Compound A

The compositions that follow are prepared according to the procedure indicated in Example 2.

Their macroscopic and microscopic appearance at T0 are studied so as to check for the absence of recrystallization of the active agent, and the pH is measured.

Compound A was assayed by HPLC. The results are expressed as percentages relative to the initial value obtained at T0.

The physical and chemical stability are evaluated at 1, 2, 3 and 6 months, at room temperature (RT) and at 40° C.

The results obtained are given after each composition.

ND=not done

i) Composition 3:

| Phase | | Ingredients | | Content (% m/m) | |
|---|---|---|---|---|---|
| A | | Steareth-10 | | 1.25 | |
| A | | Stearic acid | | 0.85 | |
| A | | Stearyl alcohol | | 0.25 | |
| A | | Steareth-2 | | 1.80 | |
| A | | Capric/caprylic acid triglycerides | | 15.50 | |
| A | | Phenoxyethanol | | 1.00 | |
| A | | DL-α-Tocopherol | | 0.20 | |
| A | | Compound A | | 0.01 | |
| B | | Cyclopentasiloxane | | 4.00 | |
| B | | Disodium edetate | | 0.10 | |
| B | | Glycerol | | 5.00 | |
| B | | Purified water | | 35.46 | |
| B | | Triethanolamine | | 0.20 | |
| B | | Benzyl alcohol | | 0.40 | |
| B | | Benzalkonium chloride | | 0.08 | |
| C | | Acrylamide/Sodium | | 4.00 | |
| | | acryloyldimethyltaurate | | | |
| | | copolymer & Isohexadecane & | | | |
| | | Polysorbate 80 | | | |
| C | | Purified water | | 30.00 | |
| CHARACTERIZATION | | MACROSCOPIC | | Thick white | |
| At T0 | | APPEARANCE | | glossy cream | |
| | | MICROSCOPIC | | Fine homogeneous | |
| | | APPEARANCE | | emulsion carpet/absence | |
| | | | | of active agent crystals | |
| | | pH | | 7.71 | |
| | | CENTRIFUGATION | | Complies | |
| | | (5000 RPM/30 min) | | | |
| Monitoring of stabilities | | 1 MONTH | 2 MONTHS | 3 MONTHS | 6 MONTHS |
| pH RT/40° C. | | 7.73/7.60 | 7.72/7.43 | 7.77/7.21 | 7.72/6.97 |
| Assay (% relative | RT/40° C. | 101.3/100.9 | 99.2/99.0 | 100.6/99.2 | 100.4/97.0 |
| active agent/T0) | | | | | |

Active agent = compound A

ii) Composition 4

| Phase | Ingredients | Content (% m/m) |
|---|---|---|
| A | Steareth-10 | 1.25 |
| A | Stearic acid | 0.85 |
| A | Stearyl alcohol | 0.25 |
| A | Steareth-2 | 1.80 |
| A | Capric/caprylic acid triglycerides | 15.50 |
| A | Phenoxyethanol | 1.00 |
| A | DL-α-Tocopherol | 0.20 |
| A | Compound A | 0.01 |
| A | Cyclopentasiloxane | 4.00 |
| B | Glycerol | 5.00 |
| B | Benzalkonium chloride | 0.08 |
| B | Purified water | 21.80 |
| B | Triethanolamine | 0.20 |
| C | Acrylamide/Sodium | 4.00 |
| | acryloyldimethyltaurate | |
| | copolymer & Isohexadecane & | |
| | Polysorbate 80 | |
| C | Purified water | 43.66 |
| C | Benzyl alcohol | 0.40 |
| CHARACTERIZATION | MACROSCOPIC | Thick white |
| At T0 | APPEARANCE | glossy cream |
| | MICROSCOPIC | Fine homogeneous |
| | APPEARANCE | emulsion carpet/absence |
| | | of active agent crystals |
| | pH | 7.71 |
| | CENTRIFUGATION | Complies |
| | (5000 RPM/30 min) | |

| Monitoring of stabilities | | 1 MONTH | 2 MONTHS | 3 MONTHS | 6 MONTHS |
|---|---|---|---|---|---|
| pH RT/40° C. | | 7.73/7.60 | 7.72/7.43 | 7.77/7.21 | 7.72/6.97 |
| Assay (% relative | RT/40° C. | 101.3%/100.9% | 99.2%/99.0% | 100.6%/99.2% | 100.4%/97.0% |
| active agent/T0) | | | | | |

iii) Composition 5:

| Phase | Ingredients | Content (% m/m) |
|---|---|---|
| A | Steareth-10 | 1.12 |
| A | Stearic acid | 0.76 |
| A | Stearyl alcohol | 0.25 |
| A | Steareth-2 | 1.62 |
| A | Capric/caprylic acid triglycerides | 10.00 |
| A | Mineral oil | 4.00 |
| A | Coconut oil | 6.00 |
| A | Sweet almond oil | 8.00 |
| A | Phenoxyethanol | 1.00 |
| A | DL-α-Tocopherol | 0.20 |
| A | Compound A | 0.01 |
| B | Glycerol | 10.00 |
| B | Purified water | 31.04 |
| B | 1% Sodium hydroxide solution | 3.50 |
| B | Benzyl alcohol | 0.40 |
| B | Benzalkonium chloride | 0.10 |
| C | Acrylamide/Sodium | 2.00 |
| | acryloyldimethyltaurate | |
| | copolymer & Isohexadecane & | |
| | Polysorbate 80 | |
| C | Purified water | 20.00 |
| CHARACTERIZATION | MACROSCOPIC | Smooth thick yellowish |
| At T0 | APPEARANCE | cream |
| | MICROSCOPIC | Fine homogeneous |
| | APPEARANCE | emulsion carpet/absence |
| | | of active agent crystals |
| | pH | 7.69 |
| | CENTRIFUGATION | Complies |
| | (5000 RPM/30 min) | |

| Monitoring of stabilities | | 1 MONTH | 2 MONTHS | 3 MONTHS | 6 MONTHS |
|---|---|---|---|---|---|
| pH RT/40° C. | | 7.57/7.34 | 7.59/7.22 | 7.56/7.05 | 7.61/6.74 |
| Assay (% relative | RT/40° C. | 101.6%/100% | 101%/99.6% | 100.3%/99.7% | 100.1%/98.6% |
| active agent/T0) | | | | | |

Active agent = compound A

iv) Composition 6:

| Phase | Ingredients | Content (% m/m) |
|---|---|---|
| A | Steareth-10 | 1.12 |
| A | Stearic acid | 0.76 |
| A | Stearyl alcohol | 0.25 |
| A | Steareth-2 | 1.62 |
| A | Capric/caprylic | 10.00 |
| | acid triglycerides | |
| A | Mineral oil | 4.00 |
| A | Coconut oil | 6.00 |
| A | Sweet almond oil | 8.00 |
| A | Phenoxyethanol | 1.00 |
| A | DL-α-Tocopherol | 0.20 |
| A | Compound A | 0.03 |
| B | Glycerol | 10.00 |
| B | Purified water | 31.02 |
| B | 1% Sodium hydroxide | 3.50 |
| | solution | |
| B | Benzyl alcohol | 0.40 |
| B | Benzalkonium chloride | 0.10 |
| C | Acrylamide/Sodium | 2.00 |
| | acryloyldimethyltaurate | |
| | copolymer & | |
| | Isohexadecane & | |
| | Polysorbate 80 | |
| C | Purified water | 20.00 |
| CHARACTERI- | MACROSCOPIC | Smooth thick yellowish |
| ZATION | APPEARANCE | cream with a slight rancid |
| At T0 | | odor |
| | MICROSCOPIC | Fine homogeneous |
| | APPEARANCE | emulsion carpet/absence |
| | | of active agent crystals |
| | pH | 7.53 |
| | CENTRIFUGATION | Complies |
| | (5000 RPM/30 min) | |

v) Composition 7:

| Phase | Ingredients | Content (% m/m) |
|---|---|---|
| A | Steareth-10 | 1.25 |
| A | Stearic acid | 0.85 |
| A | Stearyl alcohol | 0.25 |
| A | Steareth-2 | 1.80 |
| A | Capric/caprylic acid | 15.00 |
| | triglycerides | |
| A | Cyclopentasiloxane | 4.00 |
| A | Phenoxyethanol | 1.00 |
| A | DL-α-Tocopherol | 0.20 |
| A | Compound A | 0.01 |
| B | Glycerol | 30.00 |
| B | Benzalkonium chloride | 0.08 |
| B | Purified water | 8.06 |
| B | 1% Sodium hydroxide | 3.50 |
| | solution | |
| C | Acrylamide/Sodium | 4.00 |
| | acryloyldimethyltaurate | |
| | copolymer & | |
| | Isohexadecane & | |
| | Polysorbate 80 | |
| C | Purified water | 30.00 |
| CHARACTERI- | MACROSCOPIC | Thick, white, |
| ZATION | APPEARANCE | shiny cream |
| At T0 | MICROSCOPIC | Fine homogeneous |
| | APPEARANCE | emulsion carpet/ |
| | | absence of active |
| | | agent crystals |
| | pH | 7.28 |
| | CENTRIFUGATION | |
| | (5000 RPM/30 min) | Complies |

vi) Composition 8:

| Phase | Ingredients | Content (% m/m) |
|---|---|---|
| A | Steareth-10 | 1.12 |
| A | Stearic acid | 0.76 |
| A | Stearyl alcohol | 0.25 |
| A | Steareth-2 | 1.76 |
| A | Capric/caprylic acid | 15.00 |
| | triglycerides | |
| A | PPG-15 stearyl ether | 8.50 |
| A | Mineral oil | 8.50 |
| A | Phenoxyethanol | 1.00 |
| A | DL-α-Tocopherol | 0.20 |
| A | Compound A | 0.01 |
| B | Glycerol | 30.00 |
| B | Benzalkonium chloride | 0.08 |
| B | Purified water | 16.82 |
| B | 1% Sodium hydroxide | 4.00 |
| | solution | |
| C | Acrylamide/Sodium | 2.00 |
| | acryloyldimethyltaurate | |
| | copolymer & | |
| | Isohexadecane & | |
| | Polysorbate 80 | |
| C | Purified water | 10.00 |
| CHARACTERI- | MACROSCOPIC | Thick white |
| ZATION | APPEARANCE | glossy cream |
| At T0 | MICROSCOPIC | Fine homogeneous |
| | APPEARANCE | emulsion carpet/absence |
| | | of active agent crystals |
| | pH | 7.84 |
| | CENTRIFUGATION | Complies |
| | (5000 RPM/30 min) | |

vii) Composition 9:

| Phase | Ingredients | Content (% m/m) |
|---|---|---|
| A | Steareth-10 | 1.12 |
| A | Stearic acid | 0.76 |
| A | Stearyl alcohol | 0.25 |
| A | Steareth-2 | 1.62 |
| A | Castor oil | 10.00 |
| A | PPG-15 stearyl ether | 10.00 |
| A | Mineral oil | 8.50 |
| A | Phenoxyethanol | 1.00 |
| A | DL-α-Tocopherol | 0.20 |
| A | Compound A | 0.01 |
| B | Glycerol | 30.00 |
| B | Benzalkonium chloride | 0.10 |
| B | Purified water | 20.44 |
| B | 1% Sodium hydroxide solution | 4.00 |
| C | Acrylamide/Sodium | 2.00 |
| | acryloyldimethyltaurate | |
| | copolymer & Isohexadecane & | |
| | Polysorbate 80 | |
| C | Purified water | 10.00 |
| CHARACTERI- | MACROSCOPIC | Thick white |
| ZATION | APPEARANCE | glossy cream |
| At T0 | MICROSCOPIC | Fine homogeneous |
| | APPEARANCE | emulsion carpet/ |
| | | absence of active |
| | | agent crystals |
| | CENTRIFUGATION | Complies |
| | (5000 RPM/30 min) | |

viii) Composition 10:

| Phase | Ingredients | Content (% m/m) |
|---|---|---|
| A | Steareth-10 | 1.25 |
| A | Stearic acid | 0.85 |
| A | Stearyl alcohol | 0.25 |
| A | Steareth-2 | 1.80 |
| A | Capric/caprylic acid triglycerides | 15.00 |
| A | Phenoxyethanol | 1.00 |
| A | DL-α-Tocopherol | 0.20 |
| A | Compound A | 0.01 |
| A | Cyclopentasiloxane | 4.00 |
| B | Glycerol | 20.00 |
| B | Benzalkonium chloride | 0.08 |
| B | Purified water | 23.06 |
| B | 1% Sodium hydroxide solution | 3.50 |
| C | Acrylamide/Sodium | 4.00 |
| | acryloyldimethyltaurate | |
| | copolymer & Isohexadecane & | |
| | Polysorbate 80 | |
| C | Purified water | 25.00 |
| CHARACTERI- | MACROSCOPIC | Thick white glossy |
| ZATION | APPEARANCE | cream |
| At T0 | MICROSCOPIC | Fine homogeneous |
| | APPEARANCE | emulsion carpet/ |
| | | absence of active |
| | | agent crystals |
| | pH | 7.34 |
| | CENTRIFUGATION | Complies |
| | (5000 RPM/30 min) | |

ix)

x)

Composition 11:

| Phase | Ingredients | Content (% m/m) |
|---|---|---|
| A | Steareth-10 | 1.25 |
| A | Stearic acid | 0.85 |
| A | Stearyl alcohol | 0.25 |
| A | Steareth-2 | 1.80 |
| A | Capric/caprylic acid triglycerides | 15.00 |
| A | Phenoxyethanol | 1.00 |
| A | DL-α-Tocopherol | 0.20 |
| A | Compound A | 0.03 |
| A | Cyclopentasiloxane | 4.00 |
| B | Glycerol | 20.00 |
| B | Benzalkonium chloride | 0.08 |
| B | Purified water | 23.04 |
| B | 1% Sodium hydroxide solution | 3.50 |
| C | Acrylamide/Sodium | 4.00 |
| | acryloyldimethyltaurate | |
| | copolymer & Isohexadecane & | |
| | Polysorbate 80 | |
| C | Purified water | 25.00 |
| CHARACTERI- | MACROSCOPIC | Thick white glossy |
| ZATION | APPEARANCE | cream |
| At T0 | MICROSCOPIC | Fine homogeneous |
| | APPEARANCE | emulsion carpet/ |
| | | absence of active |
| | | agent crystals |
| | pH | 7.25 |
| | CENTRIFUGATION | Complies |
| | (5000 RPM/30 min) | |

### EXAMPLE 6

### Study of Tolerance after a Single Application to the Ear of BALB/C Mice

The aim of the study is to evaluate the effect of a composition according to the invention on the pro-inflammatory activity associated with retinoids in general.

A single application of 20 μl of compositions 1 and 2 was administered to the ear of the mice for 7 days. Clinical observations and measurements of the mouse ear thickness, directly linked to the inflammation, are performed from day 2 up to day 19.

Various Formulations were Tested:
the oleosome formulation containing compound A in the oily liquid globules corresponding to composition 1 of Example 2,
the oleosome formulation containing compound A in the aqueous phase corresponding to composition 2 of Example 3,
the placebo oleosome formulation corresponding to the placebo formulation (without compound A) of composition 2 of Example 3.

Each formulation was compared with a group of untreated mice.

### Results:

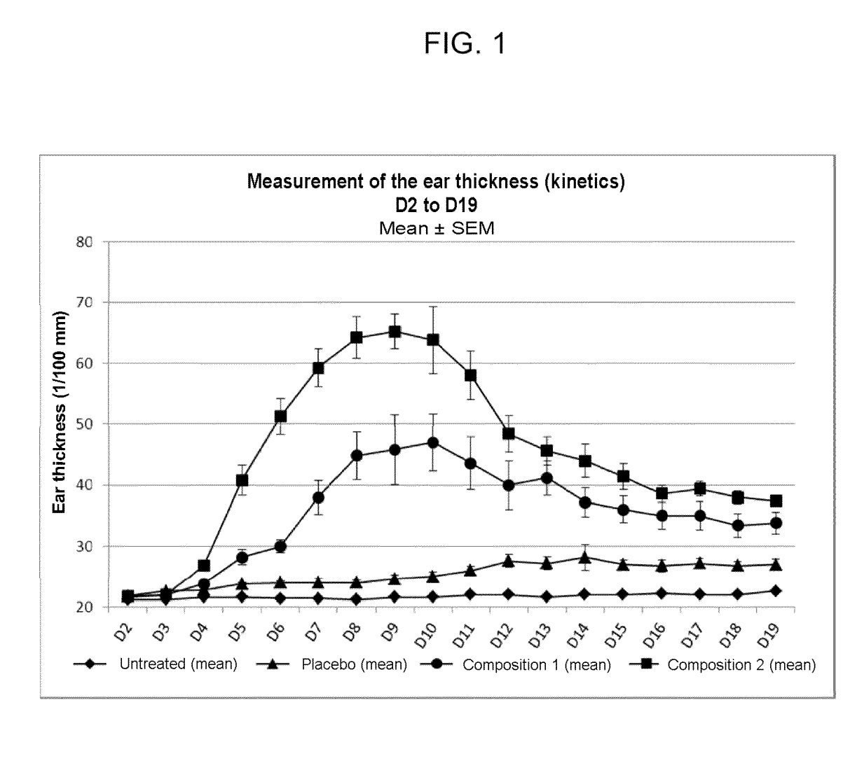

The graph in FIG. 1 describes the mouse ear thickness measurement over a period of 19 days.

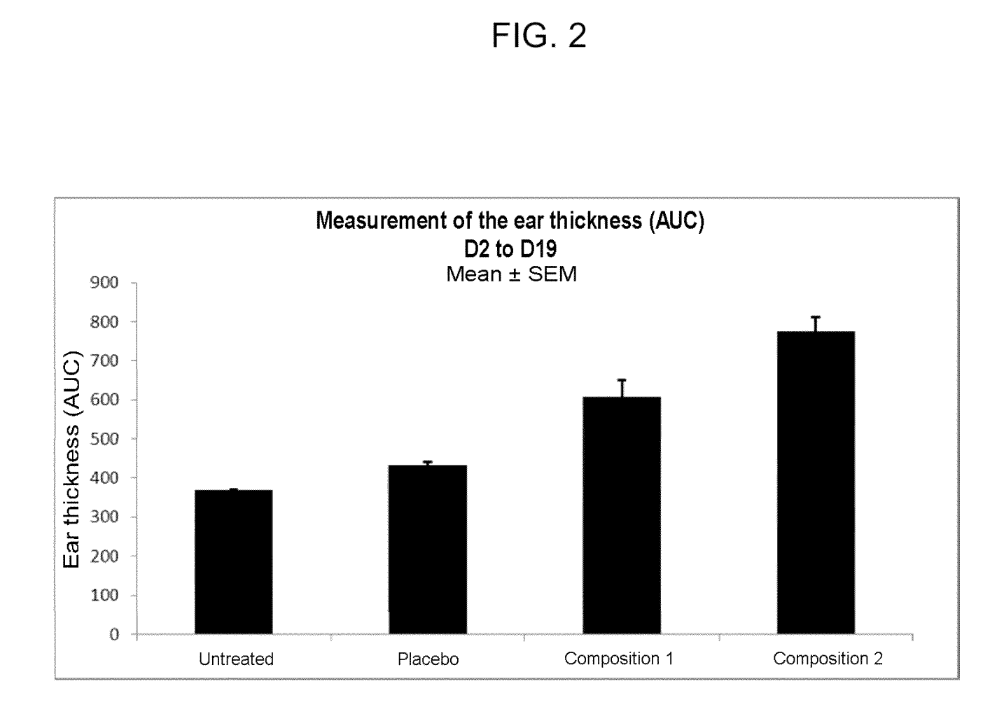

From the values in FIG. 1, the area under the curve reflecting the increase in mouse ear thickness was calculated, to obtain FIG. 2.

### Conclusion:

Even though a specific retinoid-induced irritation is observed, the oleosome formulation containing compound A in the oily liquid globules (composition 1) is less irritant than the oleosome formulation comprising compound A in the aqueous phase (composition 2).

### EXAMPLE 7

### Comparison of the In Vitro Cutaneous Release-Penetration of Compound A Formulated in a Reference Gel, with the Oleosome Formulation Comprising Compound A in the Aqueous Phase, and the Oleosome Formulation Comprising Compound A in the Oily Liquid Globules

### Conditions of the Study:

In this study, the formulations were applied for 16 h to the surface of the skin. At the end of the application, compound A is quantified in the various skin compartments: stratum corneum, epidermis, dermis and receiving liquid according to a validated bioanalysis method performed by positive electrospray ionization tandem mass spectrometry, using a Xevo machine (Waters). The quantification limit for compound A is 1 ng/ml. The LC/MS/MS conditions developed made it possible to detect up to 0.1% of the dose applied in each of the compartments (dose not absorbed, stratum, epidermis, dermis and receiving liquid).

The details of the cutaneous application are given in the table below.

Skin: 3 donors, 2 samples per donor

| | |
|---|---|
| Source | Whole abdominal human skin |
| Thickness | 0.79-1.22 mm |
| Age | 39-64 years old. |
| Franz cells | 2 cm² |
| Receiving liquid volume | 3 mL |
| Barrier function | Evaluated by determination of insensible |
| | water loss, acceptable unless contraindication |

Formulations
Reference gel containing 100 μg/g compound A
Composition 1 containing 100 μg/g compound A corresponding to Example 2
Composition 2 containing 100 μg/g compound A corresponding to Example 3
Application

| | | |
|---|---|---|
| Application | ~2 | mg/cm² |
| Amount of active agent applied | 142~241 | ng/cm² |
| Exposure time | 16 | h |

Sample assay

| Washing of donor compartment | | | | | | |
|---|---|---|---|---|---|---|
| Kleenex (for removing the | {close oversize brace} | “Excess”/Dose not absorbed | | | | |
| surplus product) | | | | | | |
| 1st strip | | | | | {close oversize brace} | Balance of masses |
| Stratum corneum | | | | | | |
| (2-15 strips max) | {close oversize brace} | Total Skin | {close oversize brace} | Total penetration | | |
| Epidermis | | | | | | |

Dermis

| | | |
|---|---|---|
| Receiving Liquid | } | Dose absorbed |

| Analyses | LC/UV and LC/MS |
|---|---|
| Quantification limit | 1 ng/ml |

The formula of the reference gel is as follows:

| Constituents | % |
|---|---|
| Compound A | 0.01 |
| Propylene glycol | 30.00 |
| Ethanol 95-96% | 67.99 |
| Klucel HF Pharma | 2.00 |

### Results:

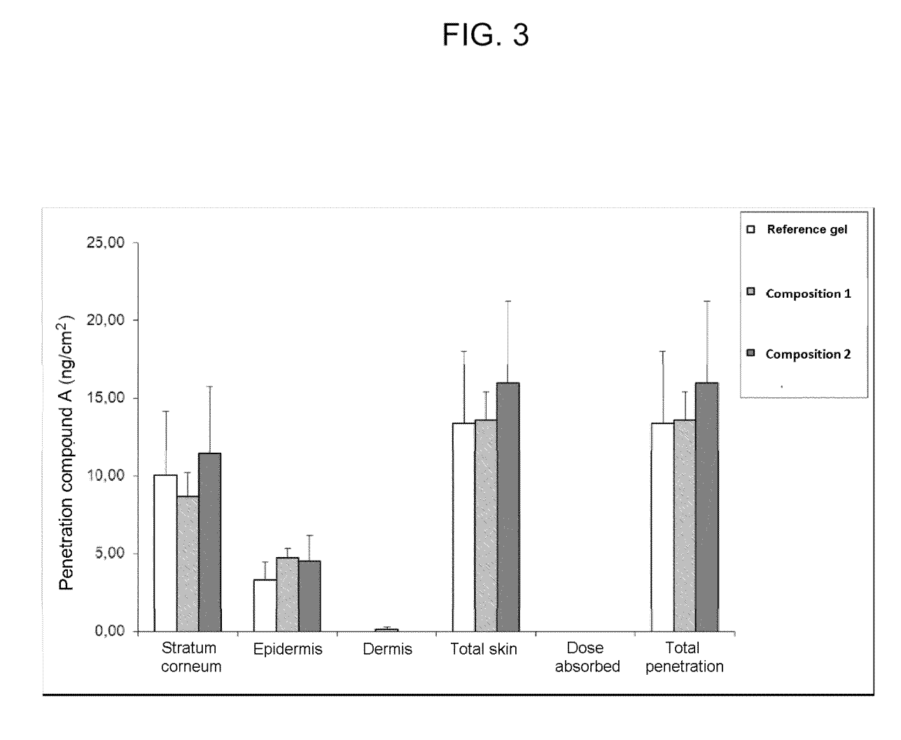

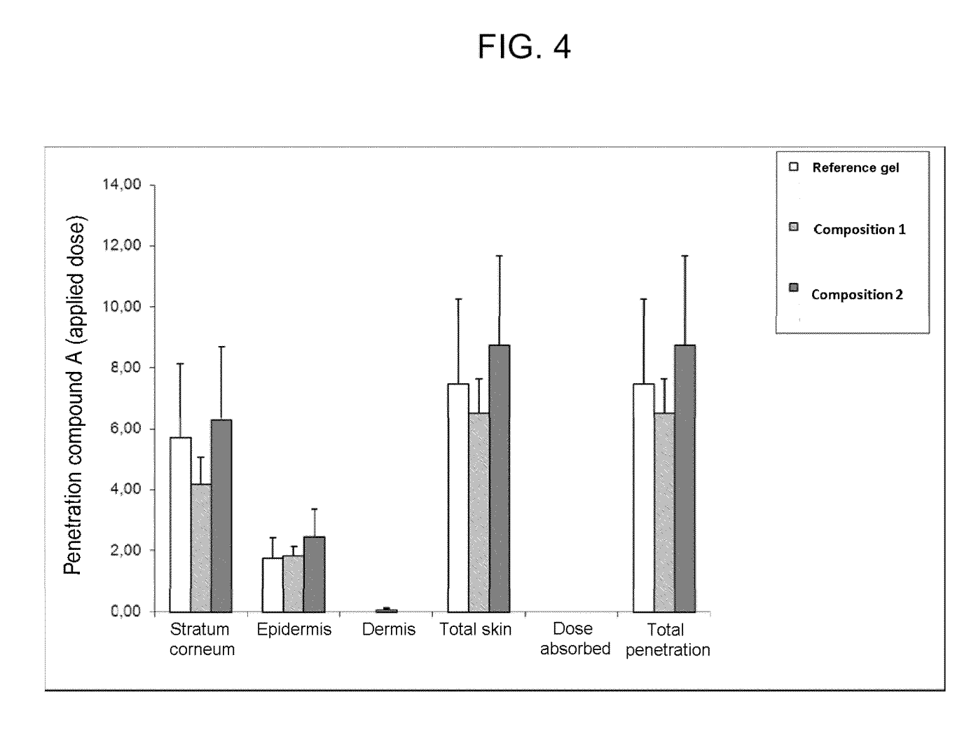

The results are presented in FIG. 3 (in ng/cm²) and in FIG. 4 (% dose applied).

### Conclusions:

Total penetration of the active agent using the various formulations is equivalent to that of the reference gel:
   For the reference gel comprising the active agent: 7.46±2.93%,
   For the oleosome formulation comprising the active agent in the oily liquid globules: 6.50±1.14% of applied dose penetrates,
   For the oleosome formulation comprising the active agent in the aqueous phase: 8.75±2.93% of applied dose penetrates.
The tissue distribution is similar irrespective of the composition:
   The largest amounts of active agent in the stratum corneum, smaller amounts in the epidermis,
   Virtually no penetration into the dermis.

Thus, surprisingly, the oleosome formulation comprising compound A in the oily liquid globules shows good penetration of the active agent, despite the internalization of the active agent and the total absence of pro-penetrating agent, versus a reference gel rich in pro-penetrating agent.

### EXAMPLE 8

### Compound A In Vitro Penetration Kinetics

The compound A penetration kinetics were studied for 24 hours, with collection times at 0.5 h, 1 h, 3 h, 6 h and 24 h. Skin biopsies were placed in diffusion cells containing phosphate buffer and the products were applied at a dose of 2 mg/cm². Six replicates (N=2 for three donors) were performed for each product and each sampling time. The same three donors were used for all the collection times.

The applied products, containing compound A, are the reference gel as defined in Example 7 above and the composition according to Example 2.

The details of the cutaneous application are given in the table below:

| Skin | 3 donors, 2 samples per donor per time, n = 6 |
|---|---|
| Source | Dermatomed abdominal human skin from a corpse |
| Thickness | 500 μm |
| Age | Not Communicated |
| Franz cells | 1-2 cm² |
| Receiving Liquid Volume | Not Communicated |
| Barrier Function | Evaluated using tritiated water |

Formulations tested
Reference gel containing 100 μg/g compound A
Composition 1 containing 100 μg/g compound A corresponding to Example 2

| | |
|---|---|
| Application of Formula | ~2 mg/cm² |
| Amount of active agent applied | Between 100-200 ng/cm² |
| Exposure time | Up to 24 hours |

Sample assay

| Exposure time | 0.5, 1, 3, 6, 24 h | | | | | |
|---|---|---|---|---|---|---|
| Washing of donor compartment | | | | | | |
| Kleenex (for removing the | {close oversize brace} | “Excess”/Dose not absorbed | | | | |
| surplus product) | | | | | | |
| 1st strip | | | | | {close oversize brace} | Balance of masses |
| Stratum corneum | | | | | | |
| (2-15 strips max) | {close oversize brace} | Total Skin | {close oversize brace} | Total penetration | | |
| Epidermis | | | | | | |

Dermis

| | | |
|---|---|---|
| Receiving Liquid | } | Dose absorbed |

| Analyses | LC/UV and LC/MS |
|---|---|
| Quantification limit | 1 ng/ml |

Two main criteria were used to classify the formulations. The penetration into the epidermis, expressed in ng/cm², was calculated, along with the area under the curve of penetration into the epidermis, calculated from time T0 to 24 hours from the values expressed in ng/cm².

The results are presented in FIG. 5.

The oleosome formulation comprising compound A in the oily liquid globules has a typical penetration profile, with a linear part followed by a plateau. However, the plateau for the cutaneous penetration of compound A was reached later than with the reference gel, thus indicating a controlled release of compound A. In addition, the penetration of compound A into the epidermis after the application of the oleosome formulation reached a value of 8.37 ng/cm², and is therefore substantially higher than that for the reference gel. Moreover, the value of the area under the curve obtained for the penetration of compound A into the epidermis is 159.8 ng/cm²*h, which is substantially close to that for the reference gel (98.06 ng/cm²*h).

### Conclusion:

These studies demonstrate that the oleosome formulation, comprising compound A in the oily liquid globules, has a penetration profile similar to that of the reference gel, allowing an exposure substantially similar to that of the reference gel. On the other hand, a relatively larger amount of compound A was detected in the epidermis after application of the oleosome formulation, indicating a preferential localization of the compound in the epidermis. Thus, the oleosome formulation comprising compound A in the oily liquid globules makes it possible to obtain controlled release of compound A, while at the same time maintaining the same total amount of penetrated active agent.

1. A composition comprising at least 3″-tert-butyl-4′-(2-hydroxyethoxy)-4″-pyrrolidin-1-yl-[1,1′;3′,1″]-terphenyl-4-carboxylic acid as pharmaceutical active agent, wherein:
the composition is an oil-in-water emulsion formed from oily liquid globules each provided with a lamellar liquid-crystal coating and dispersed in an aqueous phase,
the active agent is dissolved either in the oily liquid globules or in the aqueous phase of the oil-in-water emulsion,
each oily liquid globule is individually coated with a monolamellar or oligolamellar layer obtained from at least one lipophilic surfactant, at least one hydrophilic surfactant and at least one distinct anionic surfactant, and
the coated oily liquid globules have a mean diameter of less than 800 nm.

2. The composition as claimed in claim 1, wherein the coated oily globules have a mean diameter of less than or equal to 500 nm.

3. The composition as claimed in claim 1, wherein the lipophilic surfactant has an HLB of between 2 and 5.

4. The composition as claimed in claim 3, wherein the lipophilic surfactant with an HLB of between 2 and 5 is selected from the group consisting of sucrose distearate, diglyceryl distearate, tetraglyceryl tristearate, decaglyceryl decastearate, diglyceryl monostearate, sorbitan monostearate, sorbitan tristearate, 15 diethylene glycol monostearate, the glyceryl ester of palmitic and stearic acids, polyoxyethylene monostearate comprising 2 oxyethylene units, polyoxyethylene stearyl ether comprising 2 oxyethylene units, glyceryl monobehenate and dibehenate and pentaerythrityl tetrastearate.

5. The composition as claimed in claim 1, wherein the hydrophilic surfactant has an HLB of between 8 and 12.

6. The composition as claimed in claim 5, wherein the hydrophilic surfactant with an HLB of between 8 and 12 is selected from the group consisting of polyoxyethylene sorbitan monostearate 4 OE, polyoxyethylene sorbitan tristearate 20 OE, polyoxyethylene monostearate 8 OE, hexaglyceryl monostearate, polyoxyethylene monostearate 10 OE, polyoxyethylene stearyl ether 10 OE, polyoxyethylene distearate 12 OE and polyoxyethylene methylglucose distearate 20 OE.

7. The composition as claimed in claim 1, wherein the distinct anionic surfactant is a fatty acid or a fatty acid ester, the fatty acid comprising at least one saturated fatty chain containing more than 12 carbon atoms.

8. The composition as claimed in claim 1, wherein the distinct anionic surfactant is selected from the group consisting of stearic acid, palmitic acid, arachidic acid and behenic acid.

9. The composition as claimed in claim 1, wherein the surfactants are mixed in the following proportions to coat each oily liquid globule:
lipophilic surfactant: 45-50% by weight of the mixture;
hydrophilic surfactant: 30-35% by weight of the mixture; and
distinct anionic surfactant: 20-25% by weight of the mixture.

10. The composition as claimed in claim 1, wherein the 3″-tert-butyl-4′-(2-hydroxyethoxy)-4″-pyrrolidin-1-yl[1,1′;3′,1″]terphenyl-4-carboxylic acid is present in an amount from 0.00001% to 0.3% by weight relative to the total weight of the composition.

11. The composition as claimed in claim 1, wherein the oily liquid globules comprise at least one oily solvent selected from the group consisting of polyethoxylated fatty acids, glycol ethers, triglycerides and oils comprising the same, and fatty acid esters.

12. The composition as claimed in claim 1, wherein the ratio of the total amount of surfactants to the total amount of oily phase constituting the oily liquid globules is from 10% to 25%.

13. The composition as claimed in claim 1, wherein the 3″-tert-butyl-4′-(2-hydroxyethoxy)-4″-pyrrolidin-1-yl[1,1′;3′,1″]terphenyl-4-carboxylic acid is dissolved in the oily liquid globules.

14. The composition as claimed in claim 13, wherein the composition comprises less than 0.5% by weight of pro-penetrating agent.

15. The composition as claimed in claim 13, wherein the composition is free of pro-penetrating agent.

16. The composition as claimed in claim 1, wherein the composition is in a form suitable for topical administration.

17. The composition as claimed in claim 1, wherein the composition is provided in the form of a medicament.

18. The composition as claimed in claim 1, wherein the composition is formulated for treating dermatological complaints.

19. A method of treating dermatological complaints, the method comprising administering to an individual subject in need thereof an effective amount of the composition as claimed in claim 1, wherein the dermatological complaints are selected from the group consisting of:
1) dermatological conditions associated with a keratinization disorder relating to cell differentiation and proliferation, in particular for treating common acne, comedonal acne, polymorphic acne, acne rosacea, nodulocystic acne, acne conglobata, senile acne, secondary acne such as solar acne, acne medicamentosa or occupational acne;
2) keratinization disorders, in particular ichthyosis, ichthyosiform conditions, lamellar ichthyosis, Darier's disease, palmoplantar keratoderma, leukoplakia, pityriasis rubra pilaris and leukoplakiform conditions, cutaneous or mucosal (buccal) lichen;
3) dermatological conditions with an inflammatory immunoallergic component, with or without a cell proliferation disorder, and in particular all forms of psoriasis, whether cutaneous, mucosal or ungual, and even psoriatic arthritis, or else atopic dermatitis and the various forms of eczema;
4) skin disorders caused by exposure to UV radiation, and also for repairing or combating skin aging, whether it is photo-induced or chronological, or for reducing actinic keratoses and pigmentations, or any pathological condition associated with chronological or actinic aging, such as xerosis, pigmentations and wrinkles;
5) any condition associated with benign dermal or epidermal proliferations, whether or not they are of viral origin, such as common warts, flat warts, molluscum contagiosum and epidermodysplasia verruciformis, or oral or florid papillomatoses;
6) dermatological disorders such as immune dermatoses, for instance lupus erythematosus, bullous immune diseases and collagen diseases, such as scleroderma;
7) stigmata of epidermal and/or dermal atrophy induced by local or systemic corticosteroids, or any other form of cutaneous atrophy;
8) cicatrization disorders, or for preventing or repairing stretch marks, or else for promoting cicatrization;
9) in the treatment of any condition of fungal origin at the cutaneous level, such as tinea pedis and tinea versicolor;
10) pigmentation disorders, such as hyperpigmentation, melasma, hypopigmentation or vitiligo; and
11) cutaneous or mucosal cancerous or precancerous conditions, such as actinic keratoses, Bowen's disease, in-situ carcinomas, keratoacanthomas and skin cancers such as basal cell carcinoma (BCC), squamous cell carcinoma (SCC) and cutaneous lymphomas such as T lymphoma.

20. The method as claimed in claim 19, wherein the dermatological complaints are selected from the group consisting of: acne, ichthyosis, ichthyosiform conditions, palmoplantar hyperkeratosis and psoriasis.

21. A method of improving tolerance to 3″-tert-butyl-4′-(2-hydroxyethoxy)-4″-pyrrolidin-1-yl[1,1′;3′,1″]terphenyl-4-carboxylic acid, the method comprising administering an effective amount of the composition as claimed in claim 1 to an individual subject in need thereof.

22. A method of improving cutaneous penetration of 3″-tert-butyl-4′-(2-hydroxyethoxy)-4″-pyrrolidin-1-yl[1,1′;3′,1″]terphenyl-4-carboxylic acid, the method comprising administering to an individual subject in need thereof an effective amount of the composition as claimed in claim 1.

23. A method of obtaining controlled release of 3″-tert-butyl-4′-(2-hydroxyethoxy)-4″-pyrrolidin-1-yl[1,1′;3′,1″]terphenyl-4-carboxylic acid, the method comprising administering to an individual subject in need thereof an effective amount of the composition as claimed in claim 1, wherein the 3″-tert-butyl-4′-(2-hydroxyethoxy)-4″-pyrrolidin-1-yl[1,1;3′,1″]terphenyl-4-carboxylic acid is dissolved in the oily liquid globules.

24. A method of improving tolerance to a retinoid, the method comprising administering a composition to an individual subject in need thereof, wherein the composition comprises at least one retinoid and wherein the composition is an oil-in-water emulsion formed from oily liquid globules each comprising a lamellar liquid-crystal coating and dispersed in an aqueous phase, the retinoid being dissolved either in the oily liquid globules or in the aqueous phase of the oil-in-water emulsion, each oily liquid globule being individually coated with a monolamellar or oligolamellar layer obtained from at least one lipophilic surfactant, at least one hydrophilic surfactant and at least one distinct anionic surfactant, and the coated oily liquid globules having a mean diameter of less than 800 nm.

25. A method of improving cutaneous penetration of a retinoid, the method comprising administering to an individual subject in need thereof an effective amount of the composition as claimed in claim 1, wherein the composition does not comprise any pro-penetrating agent.

26. A method of obtaining controlled release of a retinoid, the method comprising administering to an individual subject in need thereof an effective amount of the composition as claimed in claim 1, wherein the active agent is dissolved in the oily liquid globules.

27. The composition as claimed in claim 7, wherein the fatty acid comprising at least one saturated fatty chain has between 16 and 22 carbon atoms.

28. The composition as claimed in claim 14, wherein the amount of the pro-penetrating agent is less than 0.3% by weight.

29. The composition as claimed in claim 28, wherein the amount of the pro-penetrating agent is less than 0.1% by weight.
